(19) United States
(12) Patent Application Publication
(10) Pub. No.: US 2013/0089655 A1
(43) Pub. Date: Apr. 11, 2013

(54) Device and Method for Making Replacement Heart Valve

(71) Applicant: Peter W. Gregg, Santa Cruz, CA (US)

(72) Inventor: Peter W. Gregg, Santa Cruz, CA (US)

(73) Assignee: BOSTON SCIENTIFIC SCIMED, INC., Maple Grove, MN (US)

(21) Appl. No.: 13/645,879

(22) Filed: Oct. 5, 2012

Related U.S. Application Data
(60) Provisional application No. 61/544,487, filed on Oct. 7, 2011.

(51) Int. Cl. A61F 2/24

(52) U.S. Cl. CPC A61F 2/24
USPC 427/2.25; 118/35

(57) ABSTRACT

A method and device for marking suture holes in a replacement heart valve leaflet are herein provided. The method includes providing a die having a plurality of posts, each post corresponding to suture location Ink is applied to the posts and the posts are brought into contact with the leaflet, thereby marking the locations of the suture holes on the leaflet.

### CROSS-REFERENCE TO RELATED APPLICATIONS

This Application claims priority to U.S. Provisional Application No. 61/544,487, filed Oct. 7, 2011, the entire contents of which are herein incorporated by reference.

### STATEMENT REGARDING FEDERALLY SPONSORED RESEARCH

Not Applicable

### BACKGROUND OF THE INVENTION

Repeatability in production of implantable medical devices, for example replacement heart valves, helps to ensure proper function and placement of the medical device. Various types of medical devices have components that are stitched together.

There remains a need for accurately and repeatably marking stitching locations in medical devices having such components.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF THE INVENTION

In some embodiments, method for making a replacement heart valve comprises providing a valve leaflet and a die, the die having a plurality of posts. The method further comprises applying ink to the posts, pressing the valve leaflet and the die together, contacting the posts with the valve leaflet, and marking the valve leaflet with the ink. Further, the method comprises removing the valve leaflet from the die.

In some embodiments the method further comprises providing an ink pad; the step of applying ink to the posts comprises pressing the posts into the ink pad.

In some embodiments the ink is biocompatible.

In some embodiments the method further comprises perforating the valve leaflet with the posts.

In some embodiments the posts have pointed ends.

In some embodiments the method further comprises indenting the valve leaflet with the posts.

In some embodiments the method further comprises providing a fixture and placing the valve leaflet in the fixture.

### BRIEF DESCRIPTION OF THE SEVERAL VIEWS OF THE DRAWINGS

FIG. 1 shows a perspective view of an embodiment of a device for marking valve leaflets.

FIG. 2 shows an embodiment of a replacement heart valve having three leaflets.

FIG. 3 shows an embodiment of a die for marking valve leaflets.

FIGS. 4A and 4B show an embodiment of a valve leaflet before and after marking.

FIG. 5 shows an embodiment of the device for marking valve leaflets.

FIG. 6 shows an embodiment of the die for marking and cutting valve leaflets.

FIG. 7 shows an embodiment of the device for sequentially cutting and marking valve leaflets.

### DETAILED DESCRIPTION OF THE INVENTION

While this invention may be embodied in many different forms, there are described in detail herein specific embodiments. This description is an exemplification of the principles of the invention and is not intended to limit it to the particular embodiments illustrated.

For the purposes of this disclosure, like reference numerals in the figures shall refer to like features unless otherwise indicated.

In some embodiments, a device for making a replacement heart valve comprises a die for marking stitch holes in a valve leaflet. Suture material is subsequently inserted through the stitch holes to stitch valve leaflets together, thereby forming a replacement heart valve.

In some embodiments, a device 10 comprises a die 12 and a press 14. In some embodiments, the die 12 is pushed into a leaflet 16 with press 14. Further, in some embodiments, the device 10 comprises an ink pad 18 and a table 20 on which the ink pad 18 is placed.

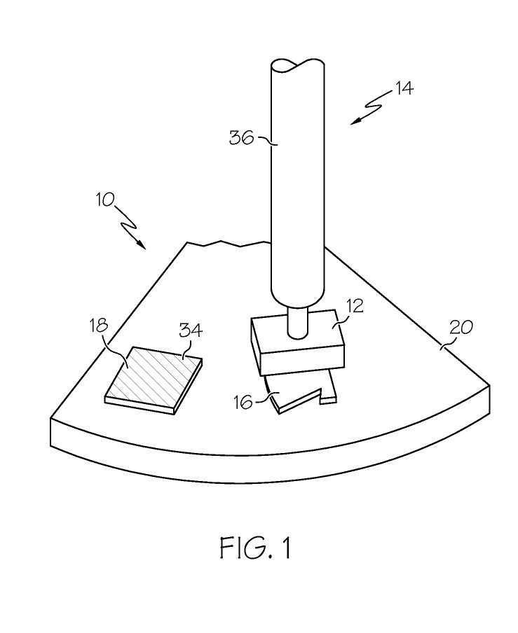

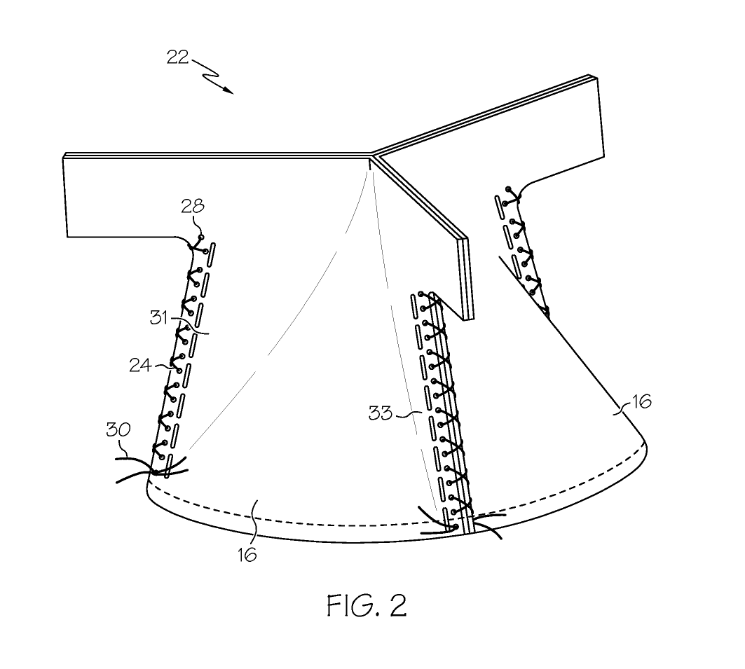

Turning to FIG. 2, a replacement heart valve 22 is shown therein having a plurality of leaflets 16 which, in some embodiments, are sewn together along sides 31 and 33. Prior to sewing the leaflets 16 together, however, the stitching pattern 24 is marked on the leaflets 16. In some embodiments, the stitching pattern 24 is marked on the leaflet 16 via die 12 (FIG. 1).

As discussed herein, the leaflet 16 is presented in various stages of manufacture. In particular, reference numeral 16 is used herein to refer to the leaflet generally, in any of the stages of manufacture. Reference numeral 16a is used herein to refer to the “leaflet blank,” from which the leaflet 16 is cut. Reference numeral 16b is used herein to refer to the “cut leaflet,” and reference numeral 16c is used herein to refer to the “marked leaflet.”

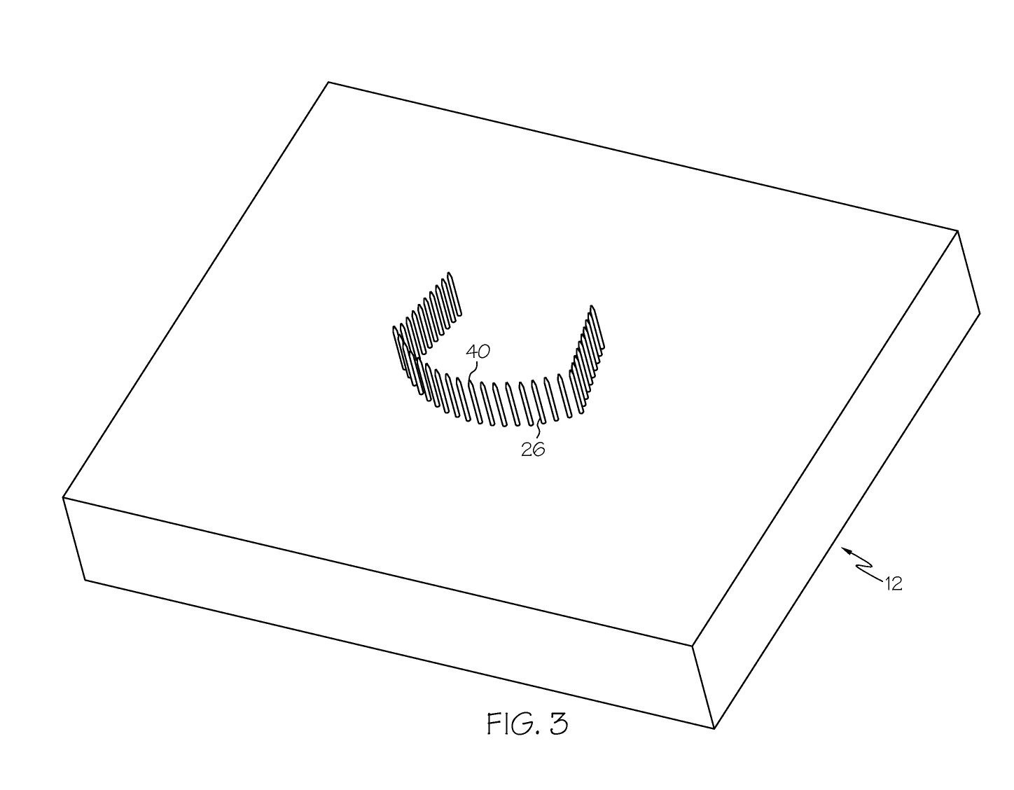

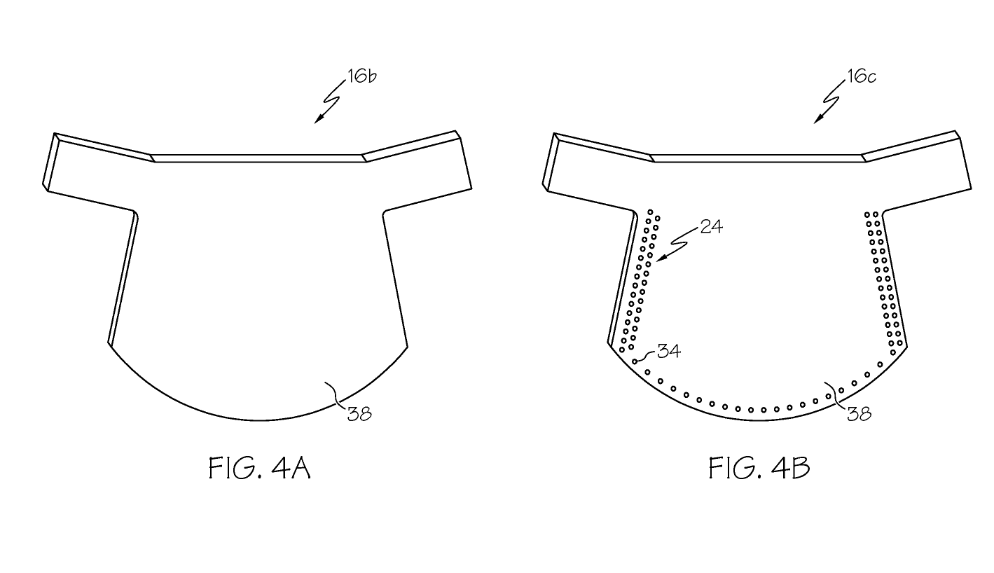

Turning to FIG. 3, the die 12 comprises a plurality of posts 26. The posts 26 correspond with stitching pattern 24 on the leaflet 16 (FIG. 2). In particular, in some embodiments, each of the posts 26 corresponds with a stitch hole 28 (FIG. 2). In this way, the stitching pattern 24 is marked on a cut leaflet 16b (FIG. 4A), via die 12 and posts 26, to produce a marked leaflet 16c (FIG. 4B). In some embodiments, the stitching pattern 24 is marked on the leaflet 16 with ink 34. In some embodiments, however, the stitching pattern 24 is marked on the leaflet 16 by indenting or perforating the leaflet 16 with the posts 26 (FIG. 3). Further, in some embodiments, the leaflet 16 is indented or perforated with posts 26 having ink thereon. In this way, the marked leaflet 16c will have stitch holes 28 through which suture material 30 can be threaded. Additionally, the leaflet material 38 immediately surrounding the stitch holes 28 will be marked with ink 34, thereby providing improved visibility for stitching.

Returning to FIG. 1, in some embodiments, the die 12 is connected to a pneumatic cylinder 36. In some embodiments, the table 20 is oriented such that the die 12 is located over ink pad 18. Subsequently, the pneumatic cylinder 36 is actuated, moving the die 12 into contact with the ink pad 18 and applying ink 34 to posts 26 (FIG. 3). The pneumatic cylinder 36 is again actuated to raise the die 12; the die 12 is relocated over the leaflet 16. In some embodiments, the table 20 is indexed to reorient the leaflet 16 and ink pad 18. The pneumatic cylinder 36 can also be reoriented.

In some embodiments, the die 12, with posts 26, is then lowered onto the cut leaflet 16b to mark, indent, and/or perforate the leaflet material 38 (FIGS. 4A, B). The die 12 is then lifted and the marked leaflet 16c is removed from the table 20.

In some embodiments, multiple cut leaflets 16b can be stacked and perforated simultaneously. Further, in some embodiments, the posts 26 comprise a pointed end 40, for example as shown in FIG. 3. In some embodiments, the posts 26 are individually placed in the die 12.

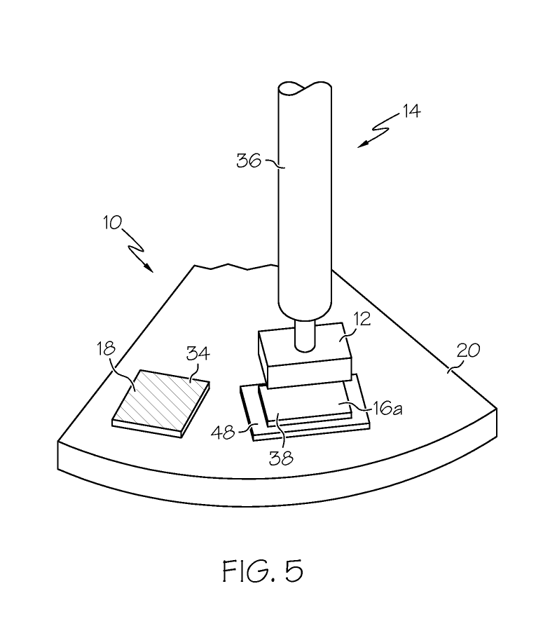

As shown in FIG. 5, in some embodiments, a leaflet blank 16a is placed on a plastic cutting pad 48. In some embodiments, for example as shown in FIG. 5, the leaflet blank 16a comprises leaflet material 38 that has not been cut into its final configuration. In this way, in some embodiments, the die 12 marks, indents, and/or perforates the leaflet blank 16a and, at the same time, cuts the leaflet blank 16a into a desired shape for assembly. Further, in some embodiments, the plastic cutting pad 48 comprises a flat sheet of soft plastic material that does not dull the die 12 as the die 12 cuts and/or perforates the leaflet 16.

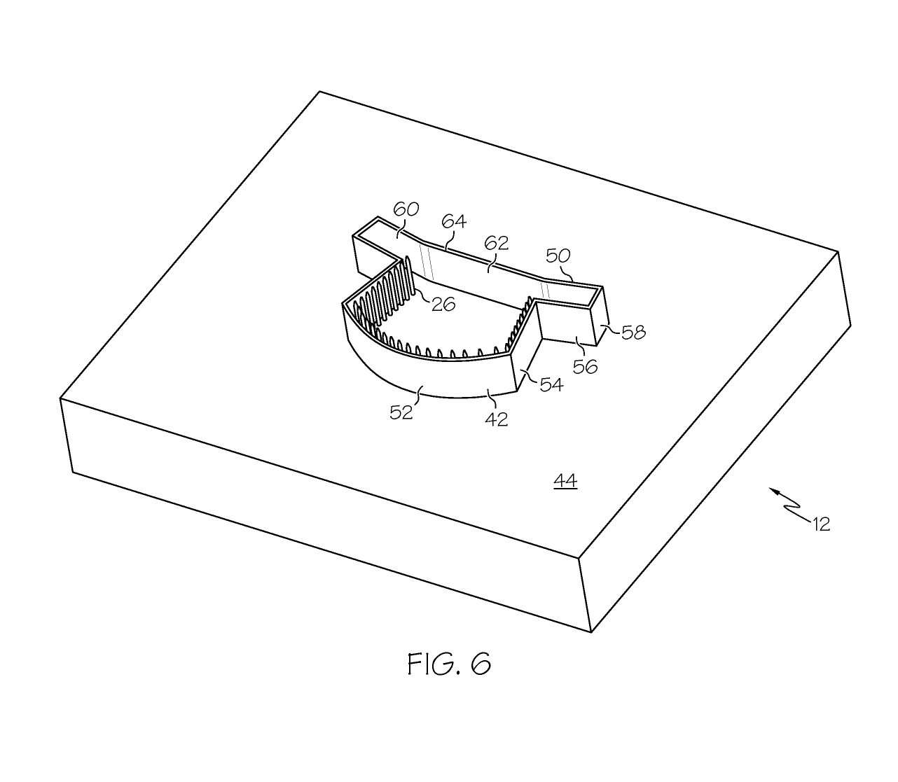

Turning to FIG. 6, an example of a die 12 having a leaflet cutter 42 and posts 26 is shown therein. The die 12 having a leaflet cutter 42 and posts 26 can simultaneously cut and mark or perforate the leaflet 16. In some embodiments, the posts 26 are spring loaded so they can recess into the die base 44. In this way, the posts 26 retract, at least partially, into the die base 44 as the leaflet cutter 42 is lowered onto the leaflet blank 16a (FIG. 5). Retraction of the posts 26 during cutting of the leaflet blank 16a helps to prevent damage to the posts 26. As further shown in FIG. 6, in some embodiments, the leaflet cutter 42 comprises a perimeter 50 having a curved bottom edge 52, two opposed inwardly extending side edges 54, two lower sleeve edges 56, two sleeve end edges 58, two upper sleeve edges 60, and a flat top edge 62. Additionally, as shown in FIG. 6, the leaflet cutter 42 comprises a cutting surface 64 along the perimeter 50. In some embodiments, the cutting surface 64 is sharpened, having a beveled edge or the like.

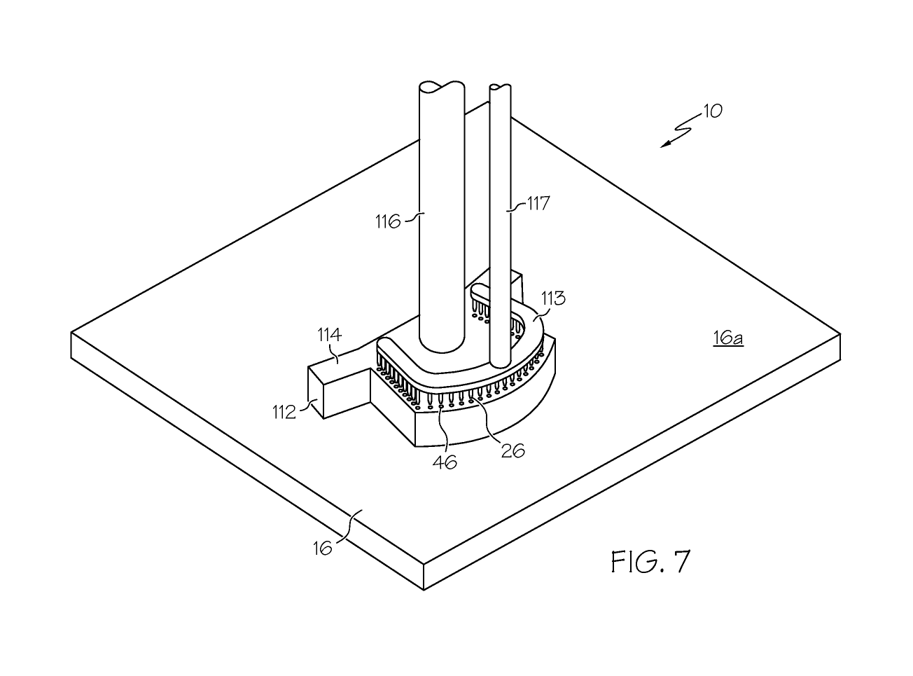

With reference to FIG. 7, in some embodiments the device 10 comprises a cutting die 112 having a backing plate 114. In some embodiments, the backing plate 114 has a plurality of holes 46 therein. Further, the cutting die 112 is attached to a first rod 116, which, in some embodiments, is connected to a pneumatic cylinder, for example as discussed with respect to FIG. 1. Additionally, in some embodiments, the device 10 comprises a marking die 113, having posts 26 for marking the leaflet 16. The marking die 113 is attached to a second rod 117. And, in some embodiments, the second rod 117 is attached to a pneumatic cylinder.

In some embodiments, the cutting die 112 is first lowered onto the leaflet blank 16a to cut the leaflet 16. Subsequently, the marking die 113 is lowered and the posts 26 are inserted through the holes 46, thereby marking the leaflet 16 with ink 34 (FIG. 1). Subsequently, the cutting die 112 and the marking die 113 are raised and the marked leaflet 16c is removed.

In some embodiments, the leaflet blank 16a is cut into shape before being placed on the table 20.

In some embodiments, the ink 34 is biocompatible, for example a food grade ink. In some embodiments, the ink 34 is Guideline Printing Blue Ink by Caloric Color in Garfield, N.J. Guideline Printing Blue Ink is comprised of:

### (TFE 461 Ceramic Blue) 60105700 Ammonium Perfluorooctanoate (CAS 3825-26-1);

### CI Pigment Blue 28 (CAS 1345-16-0);

### Cobalt Aluminate Spinel (CAS 1345-16-0);

### Octyl or Nonyl Phenoxypolyethoxyethanol (CAS 9002-93-1 or 9016-45-9);

### Polytetrafluoroethylene (CAS 9002-84-0);

### Surfactant (CAS 9016-45-9) Trace Amounts

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

What is claimed is:

1. A method of making a replacement heart valve comprising:
providing a die, the die having a plurality of posts;
providing a valve leaflet;
applying ink to the posts;
pressing the valve leaflet and the die together;
contacting the posts with the valve leaflet;
marking the valve leaflet with the ink; and
removing the valve leaflet from the die.

2. The method of claim 1 further comprising providing an ink pad, wherein the step of applying ink to the posts comprises pressing the posts into the ink pad.

3. The method of claim 1, wherein the ink is biocompatible.

4. The method of claim 1 further comprising perforating the valve leaflet with the posts.

5. The method of claim 1, wherein each of the posts comprises a pointed end.

6. The method of claim 5 further comprising indenting the valve leaflet with the posts.

7. The method of claim 1 further comprising providing a fixture and placing the valve leaflet in the fixture.

8. A method of making a replacement heart valve comprising:
providing a cutting die and a marking die, the cutting die defining a plurality of holes therein, the marking die comprising a plurality of posts;
providing a leaflet blank;
cutting the leaflet blank with the cutting die;
applying ink to the posts;
inserting the posts through the holes; and
marking the cut leaflet with the ink.

9. The method of claim 8, wherein the leaflet is removed from the cutting die.

10. The method of claim 8, wherein the ink is biocompatible.

11. The method of claim 8, wherein the cutting die is attached to a first rod and the marking die is attached to a second rod.

12. The method of claim 11, wherein the first and second rods are connected to pneumatic cylinders.

13. An apparatus for forming a heart valve leaflet comprising:
a die, the die comprising a leaflet cutter and a plurality of posts disposed within the leaflet cutter, the leaflet cutter comprising a perimeter having a curved bottom edge, two opposed inwardly extending side edges, two lower sleeve edges, two sleeve end edges, two upper sleeve edges, and a flat top edge; the leaflet cutter further comprising a cutting surface disposed along the perimeter.

14. The apparatus of claim 13, wherein the posts are configured for receiving a biocompatible ink thereon.

15. The apparatus of claim 13 further comprising a pneumatic cylinder, the die attached to the pneumatic cylinder.
